(19) United States
(12) Patent Application Publication
(10) Pub. No.: US 2019/0275354 A1
(43) Pub. Date: Sep. 12, 2019

(54) Methods, compositions and apparatuses for treating hair loss

(71) Applicant: Medical Hair Restoration, PLLC, New York, NY (US)

(72) Inventor: David Rosenberg, New York, NY (US)

(21) Appl. No.: 15/998,453

(22) Filed: Aug. 15, 2018

Related U.S. Application Data
(60) Provisional application No. 62/545,842, filed on Aug. 15, 2017.

(51) Int. Cl. A61Q 7/00; A61Q 5/00; A61K 8/97; A61K 8/42; A61K 8/92; A61K 8/34

(52) U.S. Cl. CPC A61Q 7/00; A61Q 5/00; A61K 8/34; A61K 8/42; A61K 8/92; A61K 8/97

(57) ABSTRACT

Exemplary embodiments of methods and apparatuses for a composition for hair regrowth and hair growth stimulation are provided. In some exemplary embodiments, a composition is provided including approximately 40% to approximately 55% ethyl alcohol, approximately 40% to approximately 55% propylene glycol, approximately 2% to approximately 7% minoxidil, and approximately 0.005% to approximately 0.02% biotin. The composition can further include avocado oil, melatonin, aloe, lavender oil and hydroxypropyl cellulose. A method of making the composition and a roll-on applicator for applying the composition can also be provided.

### CROSS-REFERENCE TO RELATED APPLICATIONS

This application relates to and claims priority from U.S. Provisional Patent Application Ser. No. 62/545,842 filed Aug. 15, 2017, the entire disclosure of which is hereby incorporated herein by reference.

### FIELD OF THE DISCLOSURE

The present disclosure relates to exemplary embodiments of a composition for hair regrowth and hair growth stimulation, an apparatus for applying the composition, a method of using the composition and a method of making the composition.

### BACKGROUND INFORMATION

Hair loss disorders, which include hair thinning and loss, are extremely prevalent, and affecting an estimated 20-25% of the population. While more common among adult males, hair loss disorders also affect women and children. Although most hair loss disorders are not, in themselves, damaging to the health of an individual, many hair loss disorder sufferers report associated psychological problems, such as anxiety and depression. Because of the widespread prevalence of hair loss disorders and their numerous and varied causes, there are correspondingly numerous and varied ways in which to treat hair loss disorders in order to maintain, grow, and restore hair, such as topical treatments, steroid injections and plastic surgery implantation techniques. Despite the existence of numerous and varied options for treatment of hair thinning and hair loss, there remains a need in the art for a safe, reliable, and non-invasive means to treat hair loss disorders.

### SUMMARY OF EXEMPLARY EMBODIMENTS OF THE DISCLOSURE

At least some of the above described problems can be addressed by exemplary embodiments of the present disclosure. Exemplary embodiments of the present disclosure can provide for a composition for hair regrowth and hair growth stimulation.

In some exemplary embodiments, a composition can be provided comprising approximately 40% to approximately 55% ethyl alcohol, approximately 40% to approximately 55% propylene glycol, approximately 2% to approximately 7% minoxidil, and approximately 0.005% to approximately 0.02% biotin. In some exemplary embodiments, the minoxidil can be provided at approximately 5%, and the biotin can be provided at approximately 0.01%.

In some exemplary embodiments, the composition can further comprise approximately 0.3% to approximately 1% avocado oil. The avocado oil can be provided at approximately 0.5%. The composition can further comprise approximately 0.003% to approximately 0.15% melatonin, approximately 0.05% to approximately 0.15% aloe, and approximately 0.5% to approximately 5% lavender oil. The composition can further comprise approximately 0.5% to approximately 2% hydroxypropyl cellulose.

In some exemplary embodiments, a method of making a composition can be provided, comprising combining propylene glycol with ethyl alcohol to form a solution, heating the solution to approximately 55 degrees Celsius to 65 degrees Celsius, adding minoxidil to the solution, stirring the minoxidil in the solution until it is dissolved, adding biotin to the solution, and cooling the solution. In some exemplary embodiments, the ethyl alcohol can be approximately 40% to approximately 55% of the composition, the propylene glycol can be approximately 40% to approximately 55% of the composition, the minoxidil can be approximately 2% to approximately 7% of the composition, and the biotin can be approximately 0.005% to approximately 0.02% of the composition.

In some exemplary embodiments, the method can further comprise adding avocado oil to the solution before cooling the solution. The avocado oil can be approximately 0.3% to approximately 1% of the composition. The method can further comprise adding melatonin, aloe vera and lavender oil to the solution before cooling the solution. In some exemplary embodiments, the method can further comprise creating a vortex in the solution by using a vortex mixer, adding hydroxypropyl cellulose, and stirring the solution until gelling of the solution. The melatonin can be approximately 0.003% to approximately 0.15% of the composition, the aloe vera can be approximately 0.05% to approximately 0.15% of the composition, and the lavender oil can be approximately 0.5% to approximately 5% of the composition.

In some exemplary embodiments, a roll-on applicator can be provided for applying the composition, comprising a tube portion, a composition provided within the tube portion, the composition comprising approximately 40% to approximately 55% ethyl alcohol, approximately 40% to approximately 55% propylene glycol, approximately 2% to approximately 7% minoxidil, and approximately 0.005% to approximately 0.02% biotin, and a top end of the tube portion having a dispensing portion configured to dispense the composition, wherein the dispensing portion dispenses the composition when pressed against a surface.

In some exemplary embodiments, the dispensing portion can comprise a top portion having a spherical surface having an outlet to dispense the composition. The top portion can have an orifice at a topmost portion of the spherical surface configured to dispense the composition. In some exemplary embodiments, the dispensing portion can be configured to dispense the composition around an outer periphery of the spherical surface.

In some exemplary embodiments, the composition can further comprise approximately 0.3% to approximately 1% avocado oil, approximately 0.003% to approximately 0.15% melatonin, approximately 0.05% to approximately 0.15% aloe, and approximately 0.5% to approximately 5% lavender oil. In some exemplary embodiments, the minoxidil is provided at approximately 5%, the biotin is provided at approximately 0.01%, and the avocado oil is provided at approximately 0.5%.

### BRIEF DESCRIPTION OF THE DRAWINGS

The foregoing and other objects of the present disclosure will be apparent upon consideration of the following detailed description, taken in conjunction with the accompanying drawing and claims, in which like reference characters refer to like parts throughout, and in which:

FIG. 1 illustrates a roll-on applicator for use with the composition according to an exemplary embodiment of the present disclosure.

It is intended that changes and modifications can be made to the described embodiments without departing from the true scope and spirit of the subject disclosure.

### DETAILED DESCRIPTION OF EXEMPLARY EMBODIMENTS OF DISCLOSURE

The exemplary embodiments of the present disclosure can provide for methods and apparatuses for a composition for hair regrowth and hair growth stimulation. The following description of the various embodiments is merely exemplary in nature and is in no way intended to limit the scope of the disclosure, its application, or uses.

In some exemplary embodiments, the composition can contain the following active ingredients: approximately 5% minoxidil (which can range from approximately 2% to approximately 7%), approximately 0.1% melatonin (which can range from approximately 0.003% to approximately 0.15%), approximately 0.1% aloe (which can range from approximately 0.05% to approximately 0.15%), approximately 0.01% biotin (which can range from approximately 0.005% to approximately 0.02%), approximately 0.5% avocado oil (which can range from approximately 0.3% to approximately 1.0%), and approximately 1.0% lavender oil (which can range from approximately 0.5% to approximately 5%). In some exemplary embodiments, panthenol (which can range from approximately 1% to approximately 2%), polysorbate (which can range from approximately 0.5% to approximately 4%), caffeine (which can range from approximately 0.001% to approximately 0.005%), pumpkin seed extract (which can range from approximately 0.05% to approximately 2%), and saw palmetto (which can range from approximately 0.01% to approximately 1%) can be added to the composition. In some exemplary embodiments, sunblock elements can be added, such as avobenzone (approximately 3%) and/or octinoxate (approximately 2%).

Ingredients such as but not limited to hydroxypropyl cellulose (which can range from approximately 0.5% to approximately 2%), propylene glycol (which can range from approximately 40% to approximately 55%), ethyl alcohol (which can range from approximately 40% to approximately 55%) can be used for the remaining percentages of the composition.

In some exemplary embodiments, in order to prepare an approximately 9 gram composition, the following ingredients and quantities can be provided in the composition: approximately 0.45 grams of minoxidil, approximately 0.009 grams of melatonin, approximately 0.009 grams of aloe vera (e.g., freeze dried aloe vera powder), approximately 0.009 grams of biotin, approximately 0.045 mL of avocado oil, approximately 0.09 mL of lavender oil, approximately 0.18 mL of polysorbate, approximately 4.5 mL of propylene glycol, approximately 4.5 mL of ethyl alcohol and approximately 0.09 grams of hydroxypropyl cellulose. These are exemplary ingredients and approximate ranges, and the exemplary embodiments of the present disclosure are not restricted to these exact amounts, or any of these particular ingredients.

In some exemplary embodiments according to the present disclosure, various methods for preparing the composition can be provided. For example, initially, the propylene glycol can be combined with the ethyl alcohol. The amount of propylene glycol and ethyl alcohol used can be an amount that is approximately 80%-85% (83% in some exemplary embodiments) of the final volume. For example, if one wants to make 60 mL of the composition, approximately 50 mL of propylene glycol and ethyl alcohol can be used. The combination of the propylene glycol and ethyl alcohol can be heated to approximately 55 degrees Celsius to approximately 65 degrees Celsius. Minoxidil powder can be added and stirred until it is dissolved. Appropriate amounts of melatonin, aloe vera, biotin, avocado oil and lavender oil can be added to the combination, and the solution can be allowed to cool to room temperature.

A vortex can be created in the solution, e.g., by using a vortex mixer. Hydroxypropyl cellulose can be added into the vortex. Use of a strainer can help to ensure small particle size. The solution can continue to be stirred until complete gelling of the solution is observed or the viscosity has increased until the solution has become a viscous liquid. Depending on the amounts of the ingredients used, complete gelling or desired viscosity may take approximately 4-5 hours, and can be a clear composition once it is ready. Ethyl alcohol can be added until the final volume required is provided, with the solution being carefully stirred so that air bubbles are not incorporated into the composition. The composition can be stored in an air tight container.

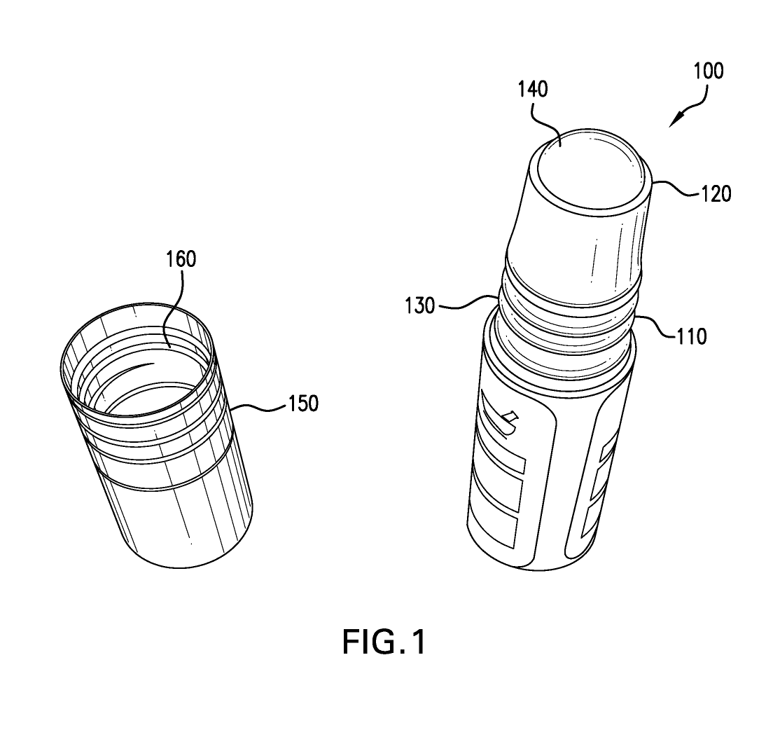

In use, the composition can be added to the scalp by pouring or applying directly on the scalp (or eyebrows or anywhere else hair is desired) from a container, and massaging the solution by hand or brush. In some exemplary embodiments, the composition can be provided in a bottle or tube having a roll on applicator on a top end. FIG. 1 illustrates a roll-on applicator 100 for use with the composition according to an exemplary embodiment of the present disclosure. The roll-on applicator 100 can have a tube portion 110, and a top end 120. The tube portion 110 can contain the composition, and can have a threaded portion 130. The top end 120 can have a dispensing portion 140. The dispensing portion 140 can be circular, but is not limited to any particular shape. A circular shape can make it easy to roll on the scalp and dispense the composition.

In some exemplary embodiments, the dispensing portion 140 can have a spherical shape in which only a top portion of dispensing portion 140 protrudes out of the tube portion 110. The spherical dispensing portion 140 can have a snug fit within the tube portion 110 so that it is locked or secured within the tube portion. In some exemplary embodiments, the dispensing portion 140 can have a small hole or orifice provided on a topmost portion of its spherical top for dispensing the composition, and can be directly applied on the scalp in the appropriate areas where the composition is being applied.

In some exemplary embodiments, the dispensing portion 140 can provide the composition around an outer periphery of the dispensing portion 140 when pressed against a user's scalp or other part of the body. When pressed against the scalp, the dispensing portion 140 can be slightly pressed inward of the tube portion 110, dispensing a small amount of the composition from the outer periphery. A mechanism, such as a spring, can be used within the tube portion 110 that presses the dispensing portion 140 upwards so that the composition is not released until a user applies pressure on the top portion of the dispensing portion 140. When a user applies slight pressure on the dispensing portion 140, pressing it within the tube portion 110, a small amount of the composition is released around the outer periphery or diameter of the spherical portion of the dispensing portion 140. A cap portion 150 can be provided having a threaded portion 160 in the interior portion of the cap portion 150 that engages with the threaded portion 130 of the tube portion 110.

The roll-on applicator 100 can have various sizes, shapes and dimensions, and can be made of various materials (such as but not limited to glass or plastic or a combination of any materials) and is not limited to any particular material, size or shape. In some exemplary embodiments, the roll-on applicator 100 can be a glass or plastic bottle approximately 2-5 inches in height. The dispensing portion 140 can be made of various materials (such as but not limited to glass, plastic, metal or a combination of any of these materials).

Various advantages can be provided based on the exemplary embodiments described above, including but not limited to ease of use and the direct delivery to the scalp rather than mixing with hair. Additionally, the composition has an appealing texture and odor compared to current market products, making it more likely a user will apply the composition. The composition can be used for growing hair on various parts of the body, including the scalp and eyebrows.

Various modifications can be provided to the exemplary embodiments described above. For example, the amount of minoxidil can be lowered as needed (e.g., to approximately 2%) if individuals are sensitive to minoxidil. Zinc can be added for those deficient in this mineral. Ultra-violet light protection can be added for those spending significant time outdoors.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure.

What is claimed is:

1. A composition comprising:
approximately 40% to approximately 55% ethyl alcohol;
approximately 40% to approximately 55% propylene glycol;
approximately 2% to approximately 7% minoxidil; and
approximately 0.005% to approximately 0.02% biotin.

2. The composition of claim 1, wherein the minoxidil is provided at approximately 5%.

3. The composition of claim 1, wherein the biotin is provided at approximately 0.01%.

4. The composition of claim 1, further comprising:
approximately 0.3% to approximately 1% avocado oil.

5. The composition of claim 4, wherein the avocado oil is provided at approximately 0.5%.

6. The composition of claim 1, further comprising:
approximately 0.003% to approximately 0.15% melatonin;
approximately 0.05% to approximately 0.15% aloe; and
approximately 0.5% to approximately 5% lavender oil.

7. The composition of claim 6, further comprising:
approximately 0.5% to approximately 2% hydroxypropyl cellulose.

8. A method of making a composition, comprising:
combining propylene glycol with ethyl alcohol to form a solution;
heating the solution to approximately 55 degrees Celsius to 65 degrees Celsius;
adding minoxidil to the solution;
stirring the minoxidil in the solution until it is dissolved;
adding biotin to the solution; and
cooling the solution.

9. The method of claim 8, wherein the ethyl alcohol is approximately 40% to approximately 55% of the composition, the propylene glycol is approximately 40% to approximately 55% of the composition, the minoxidil is approximately 2% to approximately 7% of the composition, and the biotin is approximately 0.005% to approximately 0.02% of the composition.

10. The method of claim 8, further comprising:
adding avocado oil to the solution before cooling the solution.

11. The method of claim 10, wherein the avocado oil is approximately 0.3% to approximately 1% of the composition.

12. The method of claim 10, further comprising:
adding melatonin, aloe vera and lavender oil to the solution before cooling the solution.

13. The method of claim 12, further comprising:
creating a vortex in the solution by using a vortex mixer;
adding hydroxypropyl cellulose; and
stirring the solution until gelling of the solution.

14. The method of claim 12, wherein the melatonin is approximately 0.003% to approximately 0.15% of the composition, the aloe vera is approximately 0.05% to approximately 0.15% of the composition, and the lavender oil is approximately 0.5% to approximately 5% of the composition.

15. A roll-on applicator comprising:
a tube portion;
a composition provided within the tube portion, the composition comprising approximately 40% to approximately 55% ethyl alcohol, approximately 40% to approximately 55% propylene glycol, approximately 2% to approximately 7% minoxidil, and approximately 0.005% to approximately 0.02% biotin; and
a top end of the tube portion having a dispensing portion configured to dispense the composition;
wherein the dispensing portion dispenses the composition when pressed against a surface.

16. The roll-on applicator of claim 15, wherein the dispensing portion comprises a top portion having a spherical surface having an outlet to dispense the composition.

17. The roll-on applicator of claim 16, wherein the top portion has an orifice at a topmost portion of the spherical surface configured to dispense the composition.

18. The roll-on applicator of claim 16, wherein the dispensing portion is configured to dispense the composition around an outer periphery of the spherical surface.

19. The roll-on applicator of claim 15, wherein the composition further comprises approximately 0.3% to approximately 1% avocado oil, approximately 0.003% to approximately 0.15% melatonin, approximately 0.05% to approximately 0.15% aloe, and approximately 0.5% to approximately 5% lavender oil.

20. The roll-on applicator of claim 19, wherein the minoxidil is provided at approximately 5%, the biotin is provided at approximately 0.01%, and the avocado oil is provided at approximately 0.5%.
